(19) United States
(12) Patent Application Publication
(10) Pub. No.: US 2025/0332011 A1
(43) Pub. Date: Oct. 30, 2025

(54) SYSTEMS AND METHODS MODIFYING TISSUE ADJACENT A STENT

(71) Applicant: BOSTON SCIENTIFIC SCIMED, INC., Maple Grove, MN (US)

(72) Inventor: Alexander James Lammers, Brookline, MA (US)

(73) Assignee: BOSTON SCIENTIFIC SCIMED, INC., Maple Grove, MN (US)

(21) Appl. No.: 19/188,593

(22) Filed: Apr. 24, 2025

Related U.S. Application Data
(60) Provisional application No. 63/638,630, filed on Apr. 25, 2024.

(51) Int. Cl. A61F 2/95; A61B 18/00; A61B 18/14; A61F 2/90

(52) U.S. Cl. CPC A61F 2/95; A61B 18/14; A61B 2018/00083; A61B 2018/00607; A61F 2/90; A61F 2002/9528

(57) ABSTRACT

An electrosurgical stent system includes a lumen-apposing stent including a tubular body, a proximal anchor member adjacent the proximal end, a distal anchor member adjacent the distal end, a polymeric inner liner, and a polymeric outer liner, wherein at least one portion of the outer surface of the stent is devoid of the outer liner, and an electrosurgical generator configured to energize the stent to modify tissue adjacent the at least one portion of the outer surface of the stent. The electrosurgical generator is configured to selectively operate in a tissue cutting mode and a tissue coagulation mode. A method of modifying tissue includes implanting the stent, positioning a probe adjacent the tubular body, wherein the probe is electrically coupled to the electrosurgical generator, and activating the electrosurgical generator to energize the stent to modify tissue adjacent the at least one portion of the outer surface of the stent.

### CROSS-REFERENCE TO RELATED APPLICATIONS

The application claims the benefit of U.S. Provisional Patent Application Ser. No. 63/638,630, filed on Apr. 25, 2024, the disclosure of which is incorporated herein by reference.

### TECHNICAL FIELD

The disclosure relates generally to medical devices, systems, and/or methods for modifying tissue adjacent an implant, such as a stent or endoprosthesis.

### BACKGROUND

An intraluminal prosthesis is a medical device used in the treatment of bodily lumens. One type of intraluminal prosthesis used in the repair and/or treatment of diseases in various body vessels is a stent. A stent is a generally longitudinal tubular device formed of biocompatible material which is useful to open and support various lumens in the body. For example, stents may be used in the vascular system, urogenital tract, gastrointestinal tract, esophageal tract, tracheal/bronchial tubes, and bile duct, as well as in a variety of other applications in the body. Lumen apposing stents may be used to drain pancreatic fluid collections and to provide direct biliary and gallbladder drainage. In some cases, tissue ingrowth into openings of the stent and/or tissue overgrowth over the outer surface of the stent may occur over time, making stent removal more difficult. In some cases, the stent may migrate if the lumen apposing force is insufficient or if the adjacent tissue lacks sufficient stiffness. In some cases, the stent may be placed using electrosurgical cutting to open a pathway between body lumens, which may cause bleeding. Of the known medical devices and methods, each has certain advantages and disadvantages. There is an ongoing need to provide alternative medical devices as well as alternative methods for manufacturing and using medical devices to improve hemostasis, to improve and/or ease stent removal, and/or to improve resistance to migration.

### SUMMARY

In one example, an electrosurgical stent system may comprise a lumen-apposing stent defining an inner surface and an outer surface, comprising: a tubular body formed of one or more interwoven wires and defining a lumen extending longitudinally from a proximal end to a distal end, a proximal anchor member disposed adjacent the proximal end, wherein the proximal anchor member extends radially outward from the tubular body, a distal anchor member disposed adjacent the distal end, wherein the distal anchor member extends radially outward from the tubular body, a polymeric inner liner extending along the inner surface of the lumen-apposing stent from the proximal end to the distal end, and a polymeric outer liner extending along the outer surface of the lumen-apposing stent, wherein at least one portion of the outer surface of the lumen-apposing stent is devoid of the polymeric outer liner; and an electrosurgical generator configured to energize the lumen-apposing stent to modify tissue adjacent the at least one portion of the outer surface of the lumen-apposing stent that is devoid of the polymeric outer liner.

In addition, or alternatively, to any example disclosed herein, the at least one portion of the outer surface of the lumen-apposing stent is disposed on a proximal facing surface of the proximal anchor member.

In addition, or alternatively, to any example disclosed herein, the at least one portion of the outer surface of the lumen-apposing stent is disposed between the proximal anchor member and the distal anchor member.

In addition, or alternatively, to any example disclosed herein, the at least one portion of the outer surface of the lumen-apposing stent comprises two portions of the outer surface longitudinally spaced apart from each other.

In addition, or alternatively, to any example disclosed herein, the polymeric outer liner comprises a plurality of apertures disposed between the proximal anchor member and the distal anchor member.

In addition, or alternatively, to any example disclosed herein, the at least one portion of the outer surface of the lumen-apposing stent comprises a first portion disposed on a distal facing surface of the proximal anchor member and a second portion disposed on a proximal facing surface of the distal anchor member.

In addition, or alternatively, to any example disclosed herein, the electrosurgical stent system may comprise a probe electrically coupled to the electrosurgical generator, wherein the probe is covered with an electrically insulating material.

In addition, or alternatively, to any example disclosed herein, the probe is configured to induce electrical current within the lumen-apposing stent when the probe is disposed within the lumen of the tubular body and the electrosurgical generator is active.

In addition, or alternatively, to any example disclosed herein, the probe comprises a closed loop.

In addition, or alternatively, to any example disclosed herein, the electrosurgical stent system may comprise a probe configured to couple directly to the one or more interwoven wires, the probe being electrically coupled to the electrosurgical generator.

In addition, or alternatively, to any example disclosed herein, and in a second example, an electrosurgical stent system may comprise a lumen-apposing stent defining an inner surface and an outer surface, comprising: a tubular body formed of one or more interwoven wires and defining a lumen extending longitudinally from a proximal end to a distal end, a proximal anchor member disposed adjacent the proximal end, wherein the proximal anchor member extends radially outward from the tubular body, a distal anchor member disposed adjacent the distal end, wherein the distal anchor member extends radially outward from the tubular body, a polymeric inner liner extending along the inner surface of the lumen-apposing stent from the proximal end to the distal end, and a polymeric outer liner extending along the outer surface of the lumen-apposing stent, wherein at least one portion of the outer surface of the lumen-apposing stent is devoid of the polymeric outer liner; and an electrosurgical generator configured to energize the lumen-apposing stent to modify tissue adjacent the at least one portion of the outer surface of the lumen-apposing stent that is devoid of the polymeric outer liner. The electrosurgical generator may be configured to selectively operate in a tissue cutting mode and a tissue coagulation mode.

In addition, or alternatively, to any example disclosed herein, the electrosurgical generator is configured to supply a first voltage signal to the lumen-apposing stent in the tissue coagulation mode.

In addition, or alternatively, to any example disclosed herein, in the tissue coagulation mode, the at least one portion of the outer surface of the lumen-apposing stent is configured to alter mechanical properties of tissue adjacent the at least one portion of the outer surface of the lumen-apposing stent.

In addition, or alternatively, to any example disclosed herein, the electrosurgical generator is configured to supply a second voltage signal to the lumen-apposing stent in the tissue cutting mode.

In addition, or alternatively, to any example disclosed herein, in the tissue cutting mode, the at least one portion of the outer surface of the lumen-apposing stent is configured to remove tissue adjacent the at least one portion of the outer surface of the lumen-apposing stent.

In addition, or alternatively, to any example disclosed herein, and in a third example, a method of modifying tissue adjacent a lumen-apposing stent may comprise implanting a lumen-apposing stent defining an inner surface and an outer surface at a target location, the lumen apposing stent comprising: a tubular body formed of one or more interwoven wires and defining a lumen extending longitudinally from a proximal end to a distal end, a proximal anchor member disposed adjacent the proximal end, wherein the proximal anchor member extends radially outward from the tubular body, a distal anchor member disposed adjacent the distal end, wherein the distal anchor member extends radially outward from the tubular body, a polymeric inner liner extending along the inner surface of the lumen-apposing stent from the proximal end to the distal end, and a polymeric outer liner extending along the outer surface of the lumen-apposing stent, wherein at least one portion of the outer surface of the lumen-apposing stent is devoid of the polymeric outer liner; positioning a probe adjacent the tubular body, wherein the probe is electrically coupled to an electrosurgical generator; and activating the electrosurgical generator to energize the lumen-apposing stent such that the at least one portion of the outer surface of the lumen-apposing stent that is devoid of the polymeric outer liner modifies tissue adjacent the at least one portion of the outer surface of the lumen-apposing stent.

In addition, or alternatively, to any example disclosed herein, activating the electrosurgical generator comprises activating the electrosurgical generator in a tissue cutting mode such that heat generated in the lumen-apposing stent removes tissue adjacent the at least one portion of the outer surface of the lumen-apposing stent.

In addition, or alternatively, to any example disclosed herein, activating the electrosurgical generator comprises activating the electrosurgical generator in a tissue coagulation mode such that heat generated in the lumen-apposing stent alters mechanical properties of tissue adjacent the at least one portion of the outer surface of the lumen-apposing stent.

In addition, or alternatively, to any example disclosed herein, in the tissue coagulation mode, heat generated in the lumen-apposing stent increases stiffness of tissue adjacent the at least one portion of the outer surface of the lumen-apposing stent.

In addition, or alternatively, to any example disclosed herein, in the tissue coagulation mode, heat generated in the lumen-apposing stent causes hemostasis adjacent the at least one portion of the outer surface of the lumen-apposing stent.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of a lumen-apposing stent;

FIG. 2 illustrates selected aspects of a lumen-apposing stent;

FIG. 3 illustrates selected aspects of a lumen-apposing stent;

FIG. 4 illustrates selected aspects of a lumen-apposing stent;

FIG. 5 illustrates selected aspects of a lumen-apposing stent;

FIG. 6 illustrates selected aspects of a lumen-apposing stent;

FIG. 7 is a partial cutaway view illustrating selected aspects of an electrosurgical system;

FIG. 8 is a partial cutaway view illustrating selected aspects of an electrosurgical system;

FIG. 9 illustrates selected aspects of an electrosurgical system; and

FIG. 10 is a partial cutaway view illustrating selected aspects of an electrosurgical system.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

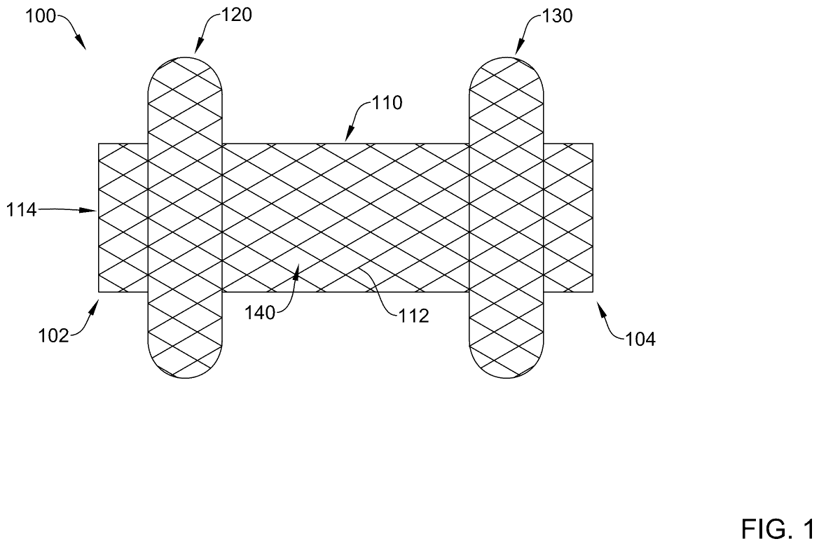

FIG. 1 illustrates selected aspects of a lumen-apposing stent 100. In some embodiments, the lumen-apposing stent 100 may define an inner surface and an outer surface. The lumen-apposing stent 100 may comprise a tubular body 110 formed of one or more interwoven wires 112. In some embodiments, the one or more interwoven wires 112 may comprise two interwoven wires, four interwoven wires, eight interwoven wires, twelve interwoven wires, 16 interwoven wires, 20 interwoven wires, 24 interwoven wires, 32 woven wires, 48 woven wires, 64 woven wires, etc. Other configurations are also contemplated. In some embodiments, the one or more interwoven 112 may have an outer diameter of about 0.003 inches (0.076 millimeters) to about 0.015 inches (0.381 millimeters), about 0.004 inches (0.102 millimeters) to about 0.012 inches (0.305 millimeters), about 0.005 inches (0.127 millimeters) to about 0.010 inches (0.254 millimeters), or another suitable size.

In at least some embodiments, the one or more interwoven wires 112 may be formed from a metallic material and/or an electrically conductive material. In some embodiments, the one or more interwoven wires 112 may be formed from a metallic composite material. For example, the one or more interwoven wires 112 may be formed from a nickel titanium alloy, a cobalt-chromium-nickel alloy, or another suitable material. In some embodiments, the tubular body 110 may be formed as a cut stent formed from a monolithic tube, a knitted stent formed from one or more filaments or wires, a braided stent formed from one or more interwoven wires, etc. Other configurations are also contemplated. In some embodiments, the lumen-apposing stent 100 may be configured to shift between a radially collapsed configuration and a radially expanded configuration. In some embodiments, the lumen-apposing stent 100 may be self-expanding, partially self-expanding, mechanically and/or balloon expandable, and/or combinations thereof. Some suitable but non-limiting examples of materials for the lumen-apposing stent 100, such as metallic materials, composite materials, combinations thereof, etc., are discussed below.

In some embodiments, the tubular body 110 may define a lumen 114 extending longitudinally from a proximal end 102 to a distal end 104. The lumen-apposing stent 100 may comprise a proximal anchor member 120 disposed adjacent the proximal end 102. In some embodiments, the proximal anchor member 120 may be disposed at the proximal end 102. The proximal anchor member 120 may extend circumferentially and/or radially outward from the tubular body 110 in the radially expanded configuration. In some embodiments, the proximal anchor member 120 may extend substantially perpendicular to a longitudinal axis of the tubular body 110 and/or the lumen-apposing stent 100. The lumen-apposing stent 100 may comprise a distal anchor member 130 disposed adjacent the distal end 104. In some embodiments, the distal anchor member 130 may be disposed at the distal end 104. The distal anchor member 130 may extend circumferentially and/or radially outward from the tubular body 110 in the radially expanded configuration. In some embodiments, the distal anchor member 130 may extend substantially perpendicular to the longitudinal axis of the tubular body 110 and/or the lumen-apposing stent 100. The proximal anchor member 120 and/or the distal anchor member 130 may have an outer extent and/or an outer diameter greater than an outer extent or an outer diameter of a portion of the tubular body 110 disposed between the proximal anchor member 120 and the distal anchor member 130.

In some embodiments, the lumen-apposing stent 100 may comprise a polymeric inner liner 140 extending along the inner surface of the lumen-apposing stent 100. In some embodiments, the polymeric inner liner 140 may be attached to the lumen-apposing stent 100 and/or the tubular body 110 at and/or adjacent the proximal end 102 and the distal end 104. In some embodiments, the polymeric inner liner 140 may be attached to the lumen-apposing stent 100 and/or the tubular body 110 only at and/or adjacent the proximal end 102 and the distal end 104. In some embodiments, the polymeric inner liner 140 may be attached to the lumen-apposing stent 100 and/or the tubular body 110 along the length of the lumen-apposing stent 100 and/or the tubular body 110. In some embodiments, the polymeric inner liner 140 may be intermittently and/or discontinuously attached to the lumen-apposing stent 100 and/or the tubular body 110 along the length of the lumen-apposing stent 100 and/or the tubular body 110. The polymeric inner liner 140 may be configured to prevent fluid leakage radially and/or laterally through the lumen-apposing stent 100 and/or the tubular body 110 (e.g., through interstices between the one or more interwoven wires 112). In some embodiments, the polymeric inner liner 140 may be configured to permit fluid flow through the lumen 114 of the lumen-apposing stent 100 and/or the tubular body 110 between the proximal end 102 and the distal end 104. Some suitable but non-limiting examples of polymeric materials for the polymeric inner liner 140 are discussed below.

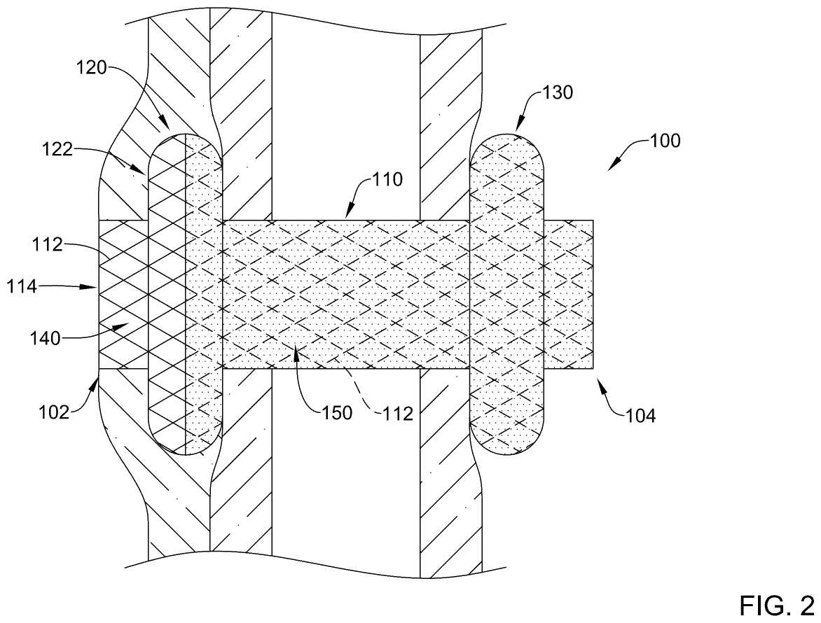

As seen in FIGS. 2-6, in some embodiments, the lumen-apposing stent 100 may comprise a polymeric outer liner 150 (shown using dotted shading) extending along the outer surface of the lumen-apposing stent 100. In some embodiments, the polymeric outer liner 150 may be attached to the lumen-apposing stent 100 and/or the tubular body 110 at and/or adjacent the proximal end 102 and the distal end 104. In some embodiments, the polymeric outer liner 150 may be attached to the lumen-apposing stent 100 and/or the tubular body 110 only at and/or adjacent the proximal end 102 and the distal end 104. In some embodiments, the polymeric outer liner 150 may be attached to the lumen-apposing stent 100 and/or the tubular body 110 along the length of the lumen-apposing stent 100 and/or the tubular body 110. In some embodiments, the polymeric outer liner 150 may be intermittently and/or discontinuously attached to the lumen-apposing stent 100 and/or the tubular body 110 along the length of the lumen-apposing stent 100 and/or the tubular body 110. In some embodiments, the polymeric outer liner 150 may be configured to prevent tissue ingrowth into the tubular body 110 and/or between and/or around the one or more interwoven wires 112 (e.g., into interstices between the one or more interwoven wires 112). Some suitable but non-limiting examples of polymeric materials for the polymeric outer liner 150 are discussed below.

In some embodiments, at least one portion of the outer surface of the lumen-apposing stent 100 may be devoid of the polymeric outer liner 150. In some embodiments, the at least one portion of the outer surface of the lumen-apposing stent 100 devoid of the polymeric outer liner 150 may be disposed on a proximal facing surface 122 of the proximal anchor member 120, as seen in FIG. 2. This configuration may be useful for treating “buried stent syndrome” or tissue overgrowth (e.g., tissue that grows over the proximal facing surface 122) to facilitate and/or ease removal of the lumen-apposing stent 100 by modifying tissue that has grown over the proximal facing surface 122 of the proximal anchor member 120. In one example, tissue that has grown over the proximal facing surface 122 of the proximal anchor member 120 may be removed by energizing the lumen-apposing stent 100 as discussed herein. Other configurations are also contemplated.

While not expressly illustrated, in some embodiments, the at least one portion of the outer surface of the lumen-apposing stent 100 devoid of the polymeric outer liner 150 may be disposed on a distal facing surface of the distal anchor member 130. This configuration may also be useful for treating “buried stent syndrome” or tissue overgrowth (e.g., tissue that grows over the distal facing surface) to facilitate and/or ease removal of the lumen-apposing stent 100 by modifying tissue that has grown over the distal facing surface of the distal anchor member 130. In one example, tissue that has grown over the distal facing surface of the distal anchor member 130 may be removed by energizing the lumen-apposing stent 100 as discussed herein. This configuration may be used independently of or in conjunction with the configuration of FIG. 2. Other configurations are also contemplated.

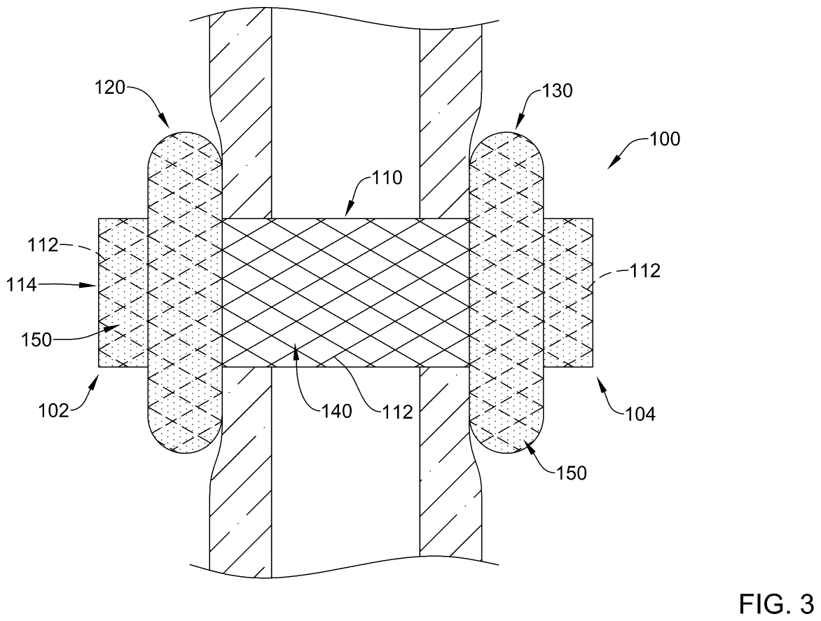

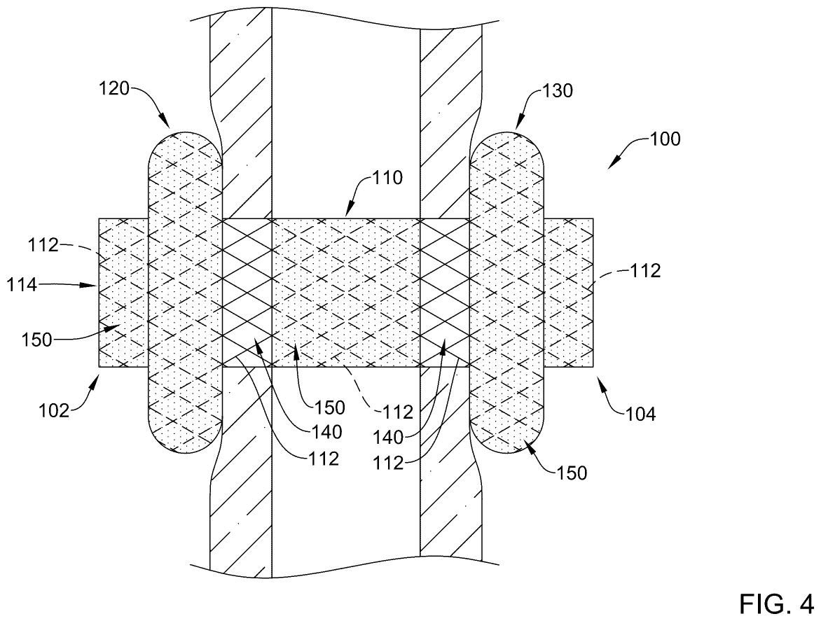

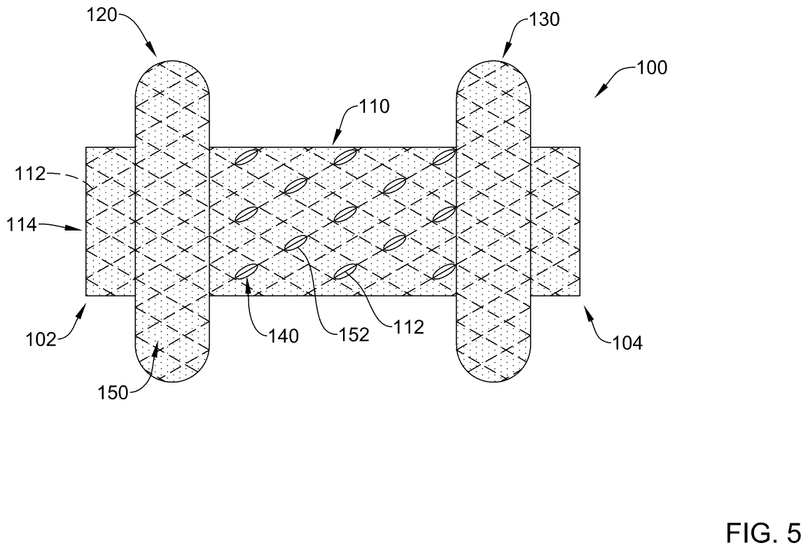

In some embodiments, the at least one portion of the outer surface of the lumen-apposing stent 100 devoid of the polymeric outer liner 150 may be disposed between the proximal anchor member 120 and the distal anchor member 130, as seen in FIGS. 3-5. In some embodiments, the at least one portion of the outer surface of the lumen-apposing stent 100 devoid of the polymeric outer liner 150 may extend continuously from the proximal anchor member 120 to the distal anchor member 130, as seen in FIG. 3. In some embodiments, the at least one portion of the outer surface of the lumen-apposing stent 100 devoid of the polymeric outer liner 150 may comprise two portions of the outer surface of the lumen-apposing stent 100 longitudinally spaced apart from each other, as seen in FIG. 4. In some embodiments, the polymeric outer liner 150 may comprise a plurality of apertures 152 disposed between the proximal anchor member 120 and the distal anchor member 130, as seen in FIG. 5, wherein the at least one portion of the outer surface of the lumen-apposing stent 100 devoid of the polymeric outer liner 150 is exposed within the plurality of apertures 152.

These configurations may be useful for modifying tissue adjacent the tubular body 110. In some embodiments, these configurations may be configured to cause hemostasis and/or coagulation in tissue adjacent the tubular body 110. In some embodiments, these configurations may be configured to cut and/or remove tissue adjacent the tubular body 110 by energizing the lumen-apposing stent 100 as discussed herein to facilitate and/or ease removal of the lumen-apposing stent 100. Other configurations are also contemplated. In some embodiments, energizing the lumen-apposing stent 100 of FIG. 3 may modify all tissue adjacent the tubular body 110 between the proximal anchor member 120 and the distal anchor member 130. In some embodiments, energizing the lumen-apposing stent 100 of FIG. 4 may focus energy to modify only tissue walls immediately adjacent the tubular body 110 between the proximal anchor member 120 and the distal anchor member 130. In some embodiments, energizing the lumen-apposing stent 100 of FIG. 5 may modify tissue adjacent the plurality of apertures 152 between the proximal anchor member 120 and the distal anchor member 130. In some embodiments, energizing the lumen-apposing stent 100 of FIG. 5 may only modify tissue immediately adjacent the plurality of apertures 152 between the proximal anchor member 120 and the distal anchor member 130. Other configurations are also contemplated.

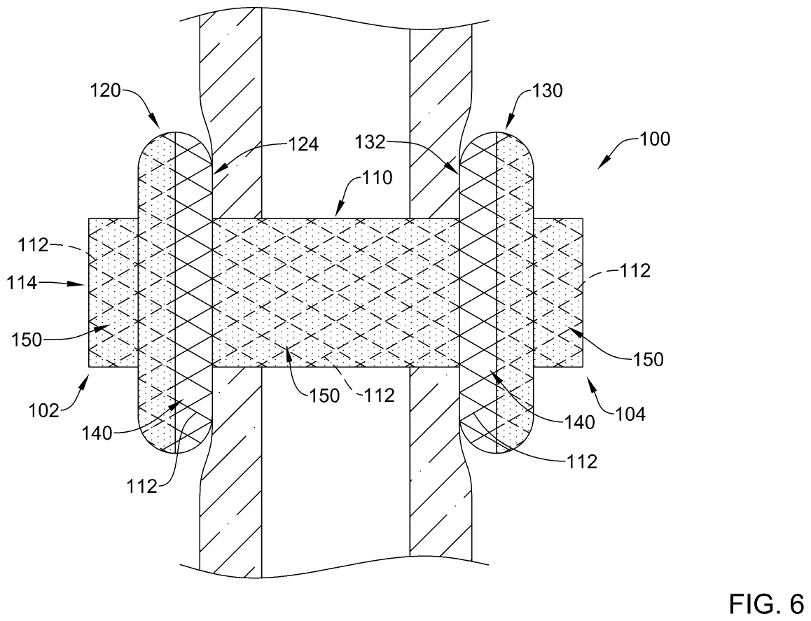

In some embodiments, the at least one portion of the outer surface of the lumen-apposing stent 100 devoid of the polymeric outer liner 150 may comprise a first portion disposed on a distal facing surface 124 of the proximal anchor member 120 and a second portion disposed on a proximal facing surface 132 of the distal anchor member 130, as seen in FIG. 6. While not expressly illustrated, in some embodiments, the at least one portion of the outer surface of the lumen-apposing stent 100 devoid of the polymeric outer liner 150 may comprise the first portion disposed on the distal facing surface 124 of the proximal anchor member 120 or the second portion disposed on the proximal facing surface 132 of the distal anchor member 130. This configuration may be useful for modifying tissue adjacent the distal facing surface 124 of the proximal anchor member 120 and/or the proximal facing surface 132 of the distal anchor member 130. In some embodiments, this configuration may be configured to cause hemostasis and/or coagulation in tissue adjacent the distal facing surface 124 of the proximal anchor member 120 and/or the proximal facing surface 132 of the distal anchor member 130. In some embodiments, this configuration may be configured to stiffen tissue adjacent the distal facing surface 124 of the proximal anchor member 120 and/or the proximal facing surface 132 of the distal anchor member 130 to increase lumen-apposing force, which may improve anchoring and/or migration resistance. Other configurations are also contemplated.

In some alternative embodiments, the one or more interwoven wires 112 may comprise a polymeric coating disposed directly thereon. In those embodiments, the polymeric coating on the one or more interwoven wires 112 may be intermittent, discontinuous, and/or strategically placed and/or omitted on the one or more interwoven wires 112 to create and/or provide discrete sections of the one of more interwoven wires 112 that are devoid of the polymeric coating (e.g., exposed). In some embodiments, the polymeric coating on the one or more interwoven wires 112 may be added prior to forming the tubular body 110 (e.g., prior to braiding the one or more interwoven wires 112 in a braided configuration, etc.) and/or prior to attaching the polymeric inner line 140 to the lumen-apposing stent 100 and/or the tubular body 110, thereby creating strategically disposed discrete sections of exposed wire along the outer surface of the lumen-apposing stent 100. In some embodiments, the discrete sections of exposed wire may permit welding at wire intersections during construction of the lumen-apposing stent 100, if desired.

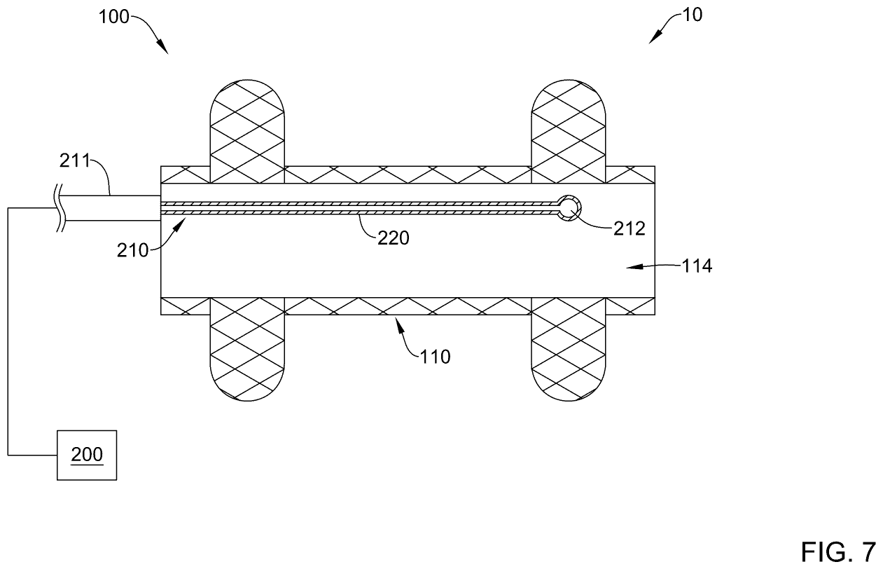

FIGS. 7-10 illustrate selected aspects of an electrosurgical system 10 comprising the lumen-apposing stent 100 and an electrosurgical generator 200 configured to energize the lumen-apposing stent 100 to modify tissue adjacent the at least one portion of the outer surface of the lumen-apposing stent 100 that is devoid of the polymeric outer liner 150. In some embodiments, the electrosurgical generator 200 may be configured to generate high frequency signals for electrosurgery. For the purpose of this disclosure, high frequency signals for electrosurgery fall within a range of about 100 kilohertz (kHz) to about 5 megahertz (M Hz). In some embodiments, high frequency signals for electrosurgery suitable for use with the current disclosure may fall within a range of about 100 kHz to about 1 M Hz, about 200 kHz to about 700 kHz, about 300 kHz to about 400 kHz, etc.

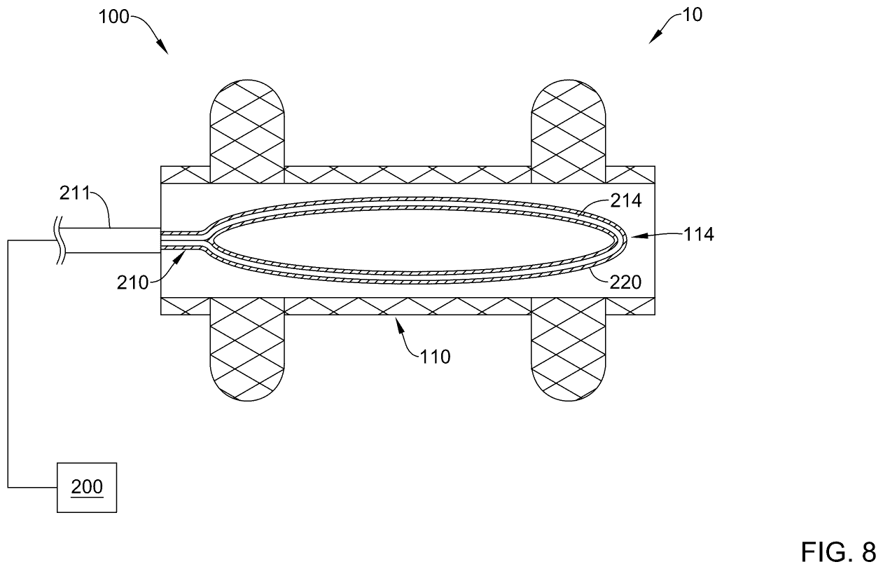

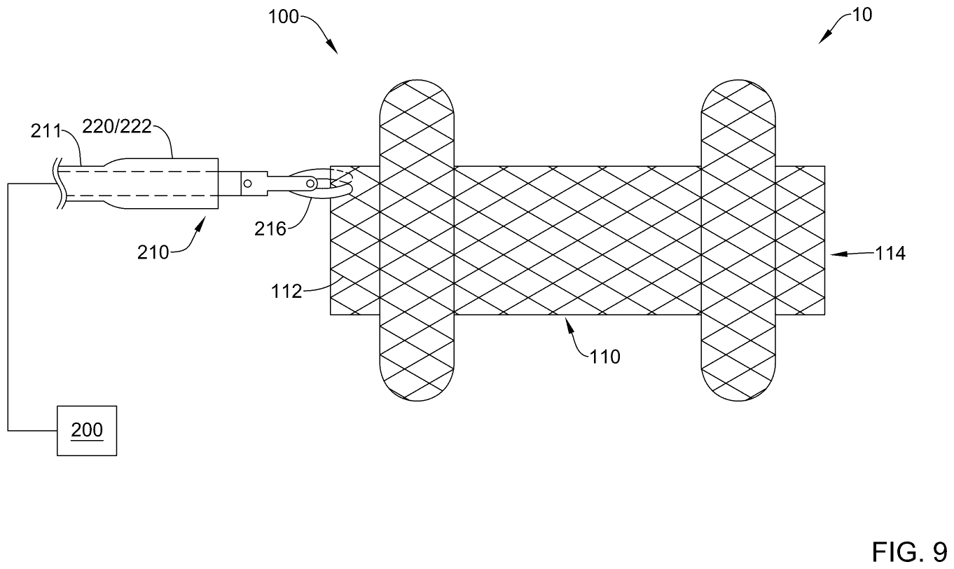

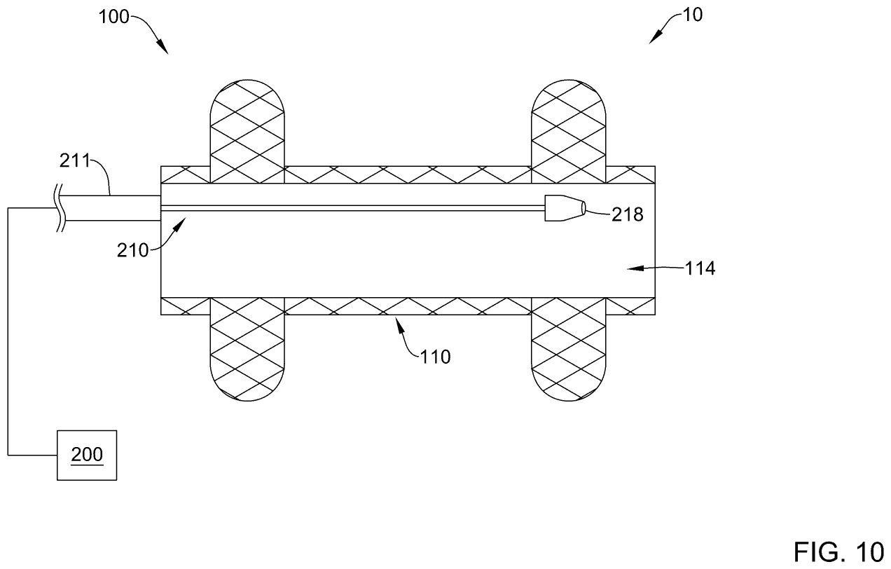

The electrosurgical system 10 may comprise a probe 210 electrically coupled to the electrosurgical generator 200. In some embodiments, the probe 210 may be covered with an electrically insulating material 220. In some embodiments, the probe 210 may be completely covered with the electrically insulating material 220, as seen in FIGS. 7-8. In some embodiments, the probe 210 may be partially covered with the electrically insulating material 220, as seen in FIGS. 9-10. In some embodiments, the probe 210 may be in electronic and/or electrical communication with the electrosurgical generator 200.

In some embodiments, the probe 210 may be configured to be translated and/or actuated in and out of an insulated sheath 211 associated with and/or used to deliver the probe 210. Upon positioning the probe 210 and/or the insulated sheath 211 at and/or adjacent the lumen-apposing stent 100 and/or the tubular body 110, the probe 210 may be advanced out of the insulated sheath 211 (or the insulated sheath 211 may be retracted proximally relative to the probe 210) to expose the probe 210 (e.g., FIG. 7). Other configurations are also contemplated.

In some embodiments, the probe 210 may configured to induce electrical current and/or resistance heating within the lumen-apposing stent 100 when the probe 210 is disposed within the lumen 114 of the tubular body 110 and the electrosurgical generator 200 is active and/or activated. In some embodiments, the probe 210 may comprise a probe tip 212, as seen in FIG. 7. In some embodiments, the probe tip 212 may have a bulbous shape. In some embodiments, the bulbous tip may be configured to concentrate high frequency energy to facilitate inducement of electrical current and/or resistance heating within the lumen-apposing stent 100 when the electrosurgical generator 200 is active and/or activated.

In some embodiments, the probe 210 may comprise a closed loop 214, as seen in FIG. 8. The closed loop 214 may permit the probe 210 to be positioned in closer proximity to the tubular body 110, thereby transmitting and/or transferring a stronger signal to the lumen-apposing stent 100 to facilitate inducement of electrical current and/or resistance heating within the lumen-apposing stent 100 when the electrosurgical generator 200 is active and/or activated. In some embodiments, the closed loop 214 may be configured to be translated and/or actuated in and out of the insulated sheath 211 associated with and/or used to deliver the probe 210. In some embodiments, when the closed loop 214 is disposed within the insulated sheath 211, the closed loop 214 may be compressed radially inward into a collapsed configuration for delivery (e.g., passage through a working channel of an endoscope, for example). Upon positioning the probe 210 and/or the insulated sheath 211 at and/or adjacent the lumen-apposing stent 100 and/or the tubular body 110, the closed loop 214 may be advanced out of the insulated sheath 211 (or the insulated sheath 211 may be retracted proximally relative to the closed loop 214) to expose the closed loop 214 and permit the closed loop 214 to expand radially toward and/or to a deployed configuration (e.g., FIG. 8). Other configurations are also contemplated.

In some embodiments, the probe 210 may comprise a clamp 216 or another direct coupling element, as seen in FIG. 9. In some embodiments, the clamp 216 may comprise an alligator clip, a spring-loaded pinching structure, an actuatable pinching structure, and the like. In some embodiments, the probe 210 and/or the clamp 216 may be configured to couple directly to the one or more interwoven wires 112 and/or the at least one portion of the outer surface of the lumen-apposing stent 100 that is devoid of the polymeric outer liner 150. In some embodiments, the probe 210 and/or the clamp 216 may be configured to directly transfer high frequency energy to the lumen-apposing stent 100 when the electrosurgical generator 200 is active and/or activated. In some embodiments, the electrically insulating material 220 may comprise an outer sheath 222 configured to advance and/or slide over the clamp 216 after coupling the clamp 216 directly to the one or more interwoven wires 112 and/or the at least one portion of the outer surface of the lumen-apposing stent 100 that is devoid of the polymeric outer liner 150 and before activating the electrosurgical generator 200. The outer sheath 222 may be configured to insulate and/or protect adjacent tissue from the clamp 216, thereby preventing unintentional tissue damage. In some embodiments, the outer sheath 222 may be coupled to and/or may be an extension of the insulated sheath 211. In some embodiments, the outer sheath 222 may be monolithically formed with the insulated sheath 211. In some embodiments, the outer sheath 222 may be separate and distinct from the insulated sheath 211. Other configurations are also contemplated.

In some embodiments, the probe 210 may comprise an exposed electrode 218 at a distal end thereof. In some embodiments, the exposed electrode 218 may be configured to ablate and/or cut tissue when the electrosurgical generator 200 is active and/or activated. In some embodiments, the probe 210 and the exposed electrode 218 may be configured to create a passageway through tissue to facilitate deployment of the lumen-apposing stent 100.

In some embodiments, the probe 210 may be configured to be translated and/or actuated in and out of the insulated sheath 211 associated with and/or used to deliver the probe 210. Upon positioning the probe 210 and/or the insulated sheath 211 at and/or adjacent the lumen-apposing stent 100 and/or the tubular body 110, the probe 210 may be advanced out of the insulated sheath 211 (or the insulated sheath 211 may be retracted proximally relative to the probe 210) to expose the probe 210 and/or the exposed electrode 218 (e.g., FIG. 10). Other configurations are also contemplated.

In some embodiments, after deploying the lumen-apposing stent 100 within the passageway through tissue, the probe 210 and the exposed electrode 218 may be positioned within the lumen 114 of the tubular body 110 adjacent the lumen-apposing stent 100 and the electrosurgical generator 200 may be activated to induce electrical current and/or resistance heating within the lumen-apposing stent 100. In some alternative embodiments, the exposed electrode 218 may be placed in direct contact with the one or more interwoven wires 112 and/or the at least one portion of the outer surface of the lumen-apposing stent 100 that is devoid of the polymeric outer liner 150 to directly transfer high frequency energy to the lumen-apposing stent 100 when the electrosurgical generator 200 is active and/or activated.

In some embodiments, the electrosurgical generator 200 may be configured to selectively operate in a tissue cutting mode and a tissue coagulation mode. Other and/or additional modes of operation are also contemplated. In some embodiments, the electrosurgical generator 200 may be configured to supply a first voltage signal to the lumen-apposing stent 100 in the tissue coagulation mode. In some embodiments, the electrosurgical generator 200 may be configured to supply a second voltage signal to the lumen-apposing stent 100 in the tissue cutting mode.

In some embodiments, when the electrosurgical generator 200 is operated in the tissue coagulation mode, the at least one portion of the outer surface of the lumen-apposing stent 100 may be configured to alter mechanical properties of tissue adjacent the at least one portion of the outer surface of the lumen-apposing stent 100. In some embodiments, when the electrosurgical generator 200 is operated in the tissue coagulation mode, heat generated in and/or by the lumen-apposing stent 100 may be configured to alter mechanical properties of tissue adjacent the at least one portion of the outer surface of the lumen-apposing stent 100. In some embodiments, when the electrosurgical generator 200 is operated in the tissue coagulation mode, heat generated in and/or by the lumen-apposing stent 100 may be configured to increase stiffness of tissue adjacent the at least one portion of the outer surface of the lumen-apposing stent 100. In some embodiments, when the electrosurgical generator 200 is operated in the tissue coagulation mode, heat generated in and/or by the lumen-apposing stent 100 may be configured to cause hemostasis adjacent the at least one portion of the outer surface of the lumen-apposing stent 100. Other configurations are also contemplated.

In some embodiments, when the electrosurgical generator 200 is operated in the tissue cutting mode, the at least one portion of the outer surface of the lumen-apposing stent 100 may be configured to remove (e.g., ablate, cut, etc.) tissue adjacent the at least one portion of the outer surface of the lumen-apposing stent 100. In some embodiments, when the electrosurgical generator 200 is operated in the tissue cutting mode, heat generated in and/or by the lumen-apposing stent 100 may be configured to remove (e.g., ablate, cut, etc.) tissue adjacent the at least one portion of the outer surface of the lumen-apposing stent 100. Other configurations are also contemplated.

In some embodiments, the electrosurgical generator 200 may be configured to generate an unmodulated sinusoidal waveform. In some embodiments, the electrosurgical generator 200 may be configured to generate a pulse-modulated sinusoidal waveform. Other configurations are also contemplated.

In some embodiments, when the electrosurgical generator 200 is operated in the tissue coagulation mode, the unmodulated sinusoidal waveform and/or the electrosurgical generator 200 may be configured to supply the first voltage signal at a maximum peak voltage of about 20 volts to about 200 volts. In some embodiments, when the electrosurgical generator 200 is operated in the tissue coagulation mode, the pulse-modulated sinusoidal waveform and/or the electrosurgical generator 200 may be configured to supply the first voltage signal at a maximum peak voltage of about 600 volts to about 1600 volts. Other voltages and/or ranges are also contemplated. In some embodiments, the electrosurgical generator 200 may be configured to supply the first voltage signal at a maximum peak voltage that when paired with a preselected surface area of the at least one portion of the outer surface of the lumen-apposing stent 100 devoid of the polymeric outer liner 150 produces a coagulating effect to the tissue in contact with the at least one portion of the outer surface of the lumen-apposing stent 100 devoid of the polymeric outer liner 150. Other voltages and/or ranges are also contemplated.

In some embodiments, the electrosurgical generator 200 may be configured to generate an unmodulated sinusoidal waveform. In some embodiments, when the electrosurgical generator 200 is operated in the tissue cutting mode, the unmodulated sinusoidal waveform and/or the electrosurgical generator 200 may be configured to supply the second voltage signal at a maximum peak voltage of about 400 volts to about 1000 volts. Other voltages and/or ranges are also contemplated. In some embodiments, the electrosurgical generator 200 may be configured to supply the first voltage signal at a maximum peak voltage that when paired with a preselected surface area of the at least one portion of the outer surface of the lumen-apposing stent 100 devoid of the polymeric outer liner 150 produces a cutting effect or an ablation effect to the tissue in contact with the at least one portion of the outer surface of the lumen-apposing stent 100 devoid of the polymeric outer liner 150. Other voltages and/or ranges are also contemplated.

In some embodiments, a method of modifying tissue adjacent a lumen-apposing stent 100 may comprise implanting the lumen-apposing stent 100 defining an inner surface and an outer surface at a target location, as seen in FIGS. 2-6 for example.

In some embodiments, the method of modifying tissue adjacent the lumen-apposing stent 100 may comprise positioning the probe 210 adjacent the tubular body 110 of the lumen-apposing stent 100, wherein the probe 210 is electrically coupled to the electrosurgical generator 200, as seen in FIGS. 7-10 for example.

In some embodiments, the method of modifying tissue adjacent the lumen-apposing stent 100 may comprise activating the electrosurgical generator 200 to energize the lumen-apposing stent 100 such that the at least one portion of the outer surface of the lumen-apposing stent 100 that is devoid of the polymeric outer liner 150 modifies tissue adjacent the at least one portion of the outer surface of the lumen-apposing stent 100. In some embodiments, activating the electrosurgical generator 200 may comprise activating the electrosurgical generator 200 in a tissue cutting mode such that heat generated in and/or by the lumen-apposing stent 100 removes tissue adjacent the at least one portion of the outer surface of the lumen-apposing stent 100. In some embodiments, activating the electrosurgical generator 200 may comprise activating the electrosurgical generator 200 in a tissue coagulation mode such that heat generated in and/or by the lumen-apposing stent 100 alters mechanical properties of tissue adjacent the at least one portion of the outer surface of the lumen-apposing stent 100. In some embodiments, in the tissue coagulation mode, heat generated in and/or by the lumen-apposing stent 100 increases stiffness of tissue adjacent the at least one portion of the outer surface of the lumen-apposing stent 100. In some embodiments, in the tissue coagulation mode, heat generated in and/or by the lumen-apposing stent 100 causes hemostasis adjacent the at least one portion of the outer surface of the lumen-apposing stent 100.

In some embodiments, the method of modifying tissue adjacent the lumen-apposing stent 100 may comprise, before positioning the lumen-apposing stent 100, forming a passageway through tissue at the target location using the probe 210. In some embodiments, forming the passageway through tissue at the target location using the probe 210 may comprise activating the electrosurgical generator in the tissue cutting mode. In some embodiments, forming the passageway through tissue at the target location using the probe 210 may comprise activating the electrosurgical generator in a third mode different from the tissue coagulation mode and the tissue cutting mode. Other configurations are also contemplated.

The materials that can be used for the various components of the electrosurgical stent system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the lumen-apposing stent, the inner line, the outer liner, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly (alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright or dark image on a fluoroscopy screen or another imaging technique (e.g., ultrasound, etc.) during a medical procedure compared to a patient's anatomy and/or other features of the system. This relatively bright or dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An electrosurgical stent system, comprising:
a lumen-apposing stent defining an inner surface and an outer surface, comprising:
a tubular body formed of one or more interwoven wires and defining a lumen extending longitudinally from a proximal end to a distal end;
a proximal anchor member disposed adjacent the proximal end, wherein the proximal anchor member extends radially outward from the tubular body;
a distal anchor member disposed adjacent the distal end, wherein the distal anchor member extends radially outward from the tubular body;
a polymeric inner liner extending along the inner surface of the lumen-apposing stent from the proximal end to the distal end; and
a polymeric outer liner extending along the outer surface of the lumen-apposing stent;
wherein at least one portion of the outer surface of the lumen-apposing stent is devoid of the polymeric outer liner; and
an electrosurgical generator configured to energize the lumen-apposing stent to modify tissue adjacent the at least one portion of the outer surface of the lumen-apposing stent that is devoid of the polymeric outer liner.

2. The electrosurgical stent system of claim 1, wherein the at least one portion of the outer surface of the lumen-apposing stent is disposed on a proximal facing surface of the proximal anchor member.

3. The electrosurgical stent system of claim 1, wherein the at least one portion of the outer surface of the lumen-apposing stent is disposed between the proximal anchor member and the distal anchor member.

4. The electrosurgical stent system of claim 3, wherein the at least one portion of the outer surface of the lumen-apposing stent comprises two portions of the outer surface longitudinally spaced apart from each other.

5. The electrosurgical stent system of claim 3, wherein the polymeric outer liner comprises a plurality of apertures disposed between the proximal anchor member and the distal anchor member.

6. The electrosurgical stent system of claim 1, wherein the at least one portion of the outer surface of the lumen-apposing stent comprises a first portion disposed on a distal facing surface of the proximal anchor member and a second portion disposed on a proximal facing surface of the distal anchor member.

7. The electrosurgical stent system of claim 1, further comprising a probe electrically coupled to the electrosurgical generator, wherein the probe is covered with an electrically insulating material.

8. The electrosurgical stent system of claim 7, wherein the probe is configured to induce electrical current within the lumen-apposing stent when the probe is disposed within the lumen of the tubular body and the electrosurgical generator is active.

9. The electrosurgical stent system of claim 8, wherein the probe comprises a closed loop.

10. The electrosurgical stent system of claim 1, further comprising a probe configured to couple directly to the one or more interwoven wires, the probe being electrically coupled to the electrosurgical generator.

11. An electrosurgical stent system, comprising:
a lumen-apposing stent defining an inner surface and an outer surface, comprising:
a tubular body formed of one or more interwoven wires and defining a lumen extending longitudinally from a proximal end to a distal end;
a proximal anchor member disposed adjacent the proximal end, wherein the proximal anchor member extends radially outward from the tubular body;
a distal anchor member disposed adjacent the distal end, wherein the distal anchor member extends radially outward from the tubular body;
a polymeric inner liner extending along the inner surface of the lumen-apposing stent from the proximal end to the distal end; and
a polymeric outer liner extending along the outer surface of the lumen-apposing stent;
wherein at least one portion of the outer surface of the lumen-apposing stent is devoid of the polymeric outer liner; and
an electrosurgical generator configured to energize the lumen-apposing stent to modify tissue adjacent the at least one portion of the outer surface of the lumen-apposing stent that is devoid of the polymeric outer liner;
wherein the electrosurgical generator is configured to selectively operate in a tissue cutting mode and a tissue coagulation mode.

12. The electrosurgical stent system of claim 11, wherein the electrosurgical generator is configured to supply a first voltage signal to the lumen-apposing stent in the tissue coagulation mode.

13. The electrosurgical stent system of claim 11, wherein in the tissue coagulation mode, the at least one portion of the outer surface of the lumen-apposing stent is configured to alter mechanical properties of tissue adjacent the at least one portion of the outer surface of the lumen-apposing stent.

14. The electrosurgical stent system of claim 11, wherein the electrosurgical generator is configured to supply a second voltage signal to the lumen-apposing stent in the tissue cutting mode.

15. The electrosurgical stent system of claim 11, wherein in the tissue cutting mode, the at least one portion of the outer surface of the lumen-apposing stent is configured to remove tissue adjacent the at least one portion of the outer surface of the lumen-apposing stent.

16. A method of modifying tissue adjacent a lumen-apposing stent, comprising:
implanting a lumen-apposing stent defining an inner surface and an outer surface at a target location, the lumen apposing stent comprising:
a tubular body formed of one or more interwoven wires and defining a lumen extending longitudinally from a proximal end to a distal end;
a proximal anchor member disposed adjacent the proximal end, wherein the proximal anchor member extends radially outward from the tubular body;
a distal anchor member disposed adjacent the distal end, wherein the distal anchor member extends radially outward from the tubular body;
a polymeric inner liner extending along the inner surface of the lumen-apposing stent from the proximal end to the distal end; and
a polymeric outer liner extending along the outer surface of the lumen-apposing stent;
wherein at least one portion of the outer surface of the lumen-apposing stent is devoid of the polymeric outer liner;
positioning a probe adjacent the tubular body, wherein the probe is electrically coupled to an electrosurgical generator; and
activating the electrosurgical generator to energize the lumen-apposing stent such that the at least one portion of the outer surface of the lumen-apposing stent that is devoid of the polymeric outer liner modifies tissue adjacent the at least one portion of the outer surface of the lumen-apposing stent.

17. The method of claim 16, wherein activating the electrosurgical generator comprises activating the electrosurgical generator in a tissue cutting mode such that heat generated in the lumen-apposing stent removes tissue adjacent the at least one portion of the outer surface of the lumen-apposing stent.

18. The method of claim 16, wherein activating the electrosurgical generator comprises activating the electrosurgical generator in a tissue coagulation mode such that heat generated in the lumen-apposing stent alters mechanical properties of tissue adjacent the at least one portion of the outer surface of the lumen-apposing stent.

19. The method of claim 18, wherein in the tissue coagulation mode, heat generated in the lumen-apposing stent increases stiffness of tissue adjacent the at least one portion of the outer surface of the lumen-apposing stent.

20. The method of claim 18, wherein in the tissue coagulation mode, heat generated in the lumen-apposing stent causes hemostasis adjacent the at least one portion of the outer surface of the lumen-apposing stent.
